(19) United States
(12) Patent Application Publication
(10) Pub. No.: US 2019/0142342 A1
(43) Pub. Date: May 16, 2019

(54) HOSPITAL BED AUTOMATION SYSTEM AND METHODS FOR PERFORMING SIGNAL READ AND CORRELATION AS WELL AS REAL-TIME DATA PROCESSING

(71) Applicant: Pulse Participações S.A., Rio De Janeiro (BR)

(72) Inventors: Gustavo de Freitas Nobre, Rio de Janeiro (BR); Marcelo Kalichsztein, Rio de Janeiro (BR); Marcelo Martínez Ramos, Rio de Janeiro (BR)

(21) Appl. No.: 16/300,272

(22) PCT Filed: May 11, 2017
(86) PCT No.: PCT/BR2017/000045

§ 371 Date: Nov. 9, 2018

(30) Foreign Application Priority Data
May 11, 2016 (BR) 10 2016 0106192

(51) Int. Cl. A61B 5/00; A61B 5/0205; A61B 5/20; A61G 7/057; G16H 10/60

(52) U.S. Cl. CPC A61B 5/6892; A61B 5/02055; A61B 5/204; A61B 5/0017; A61B 5/7425; A61B 5/021; A61B 5/746; A61B 5/7246; A61B 5/0022; A61G 7/057; G16H 10/60; A61B 5/7275

(57) ABSTRACT

The present invention relates to a hospital bed automation system that collects vital signs and other parameters of a patient, being captured from a plurality of sensors, medical equipment and devices installed at the rim of the bed, the patient and the internalization environment. The system further has microprocessors installed at the inlet of the bed and the bed of the patient for capturing the signals that are transmitted to a central server with database for recording said data from each patient after a step of reading and correlating the signals for processing the data in real time. An application server performs the interface between the database, a system for managing medical information and local and remote users accessing information. The system is established by wired and wireless communication networks by Ethernet or Serial RS232 standard, RS422 and RS485, Wifi, cellular, digital or infrared. Methods for performing read and correlation of signals for real-time data processing are performed by computer programs embedded in microprocessors. It has technical operability with varying equipment or systems and can be employed on medical centers, hospitals and home medical treatment.

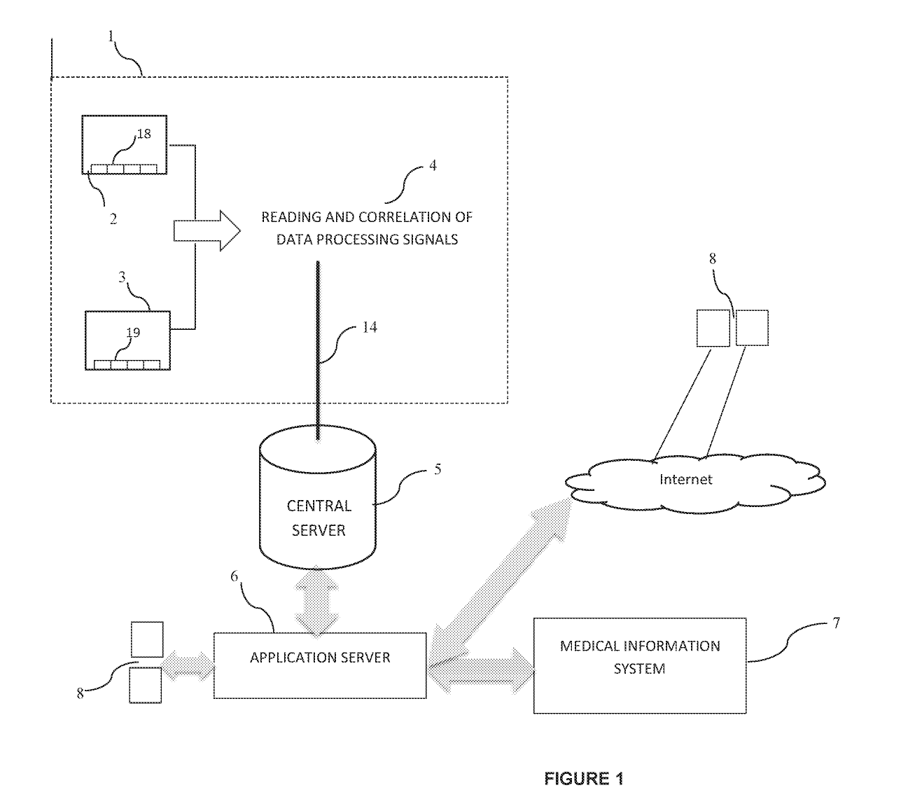

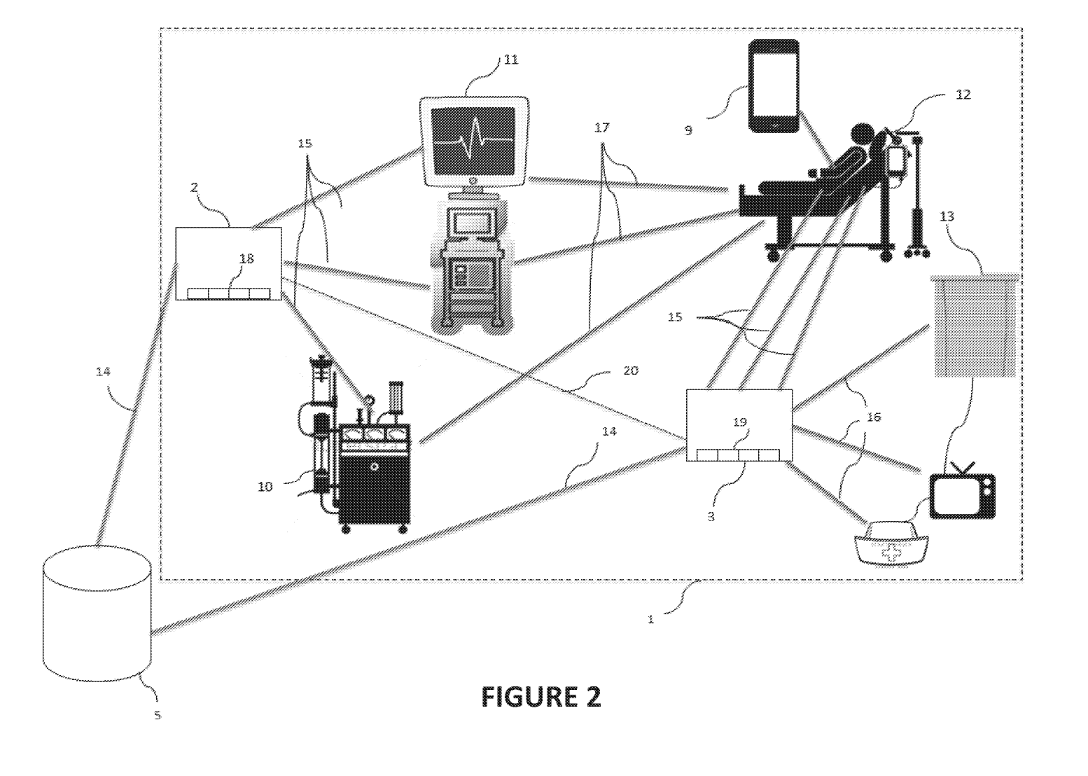

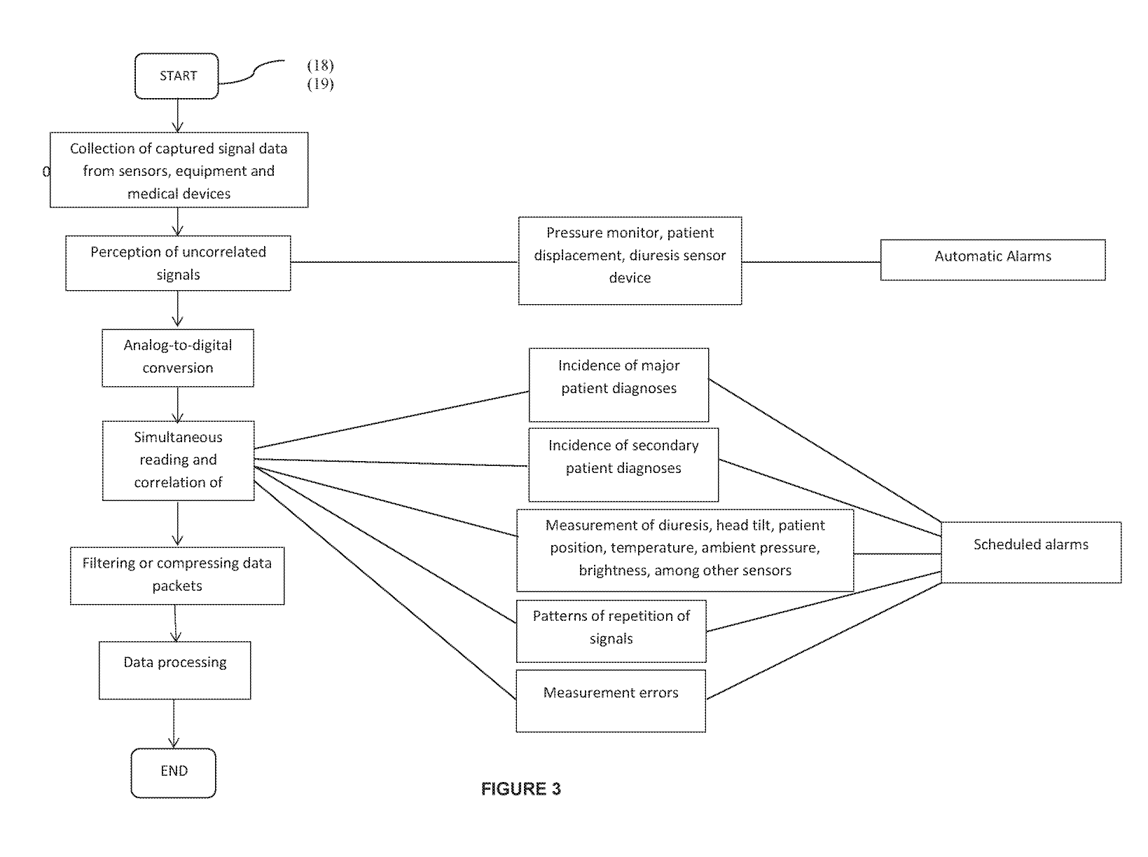

### FIELD OF THE INVENTION

The present invention relates to the technical field of computing and electronics for capturing, storing and transmitting signals, data processing and logging, especially adapted for specific applications with sensors, equipment and medical devices, in order to automate measurements, monitor, read and correlate signals from a patient, which are processed for database registration for diagnostic, tracking and treatment purposes. It is a hospital bed automation system and methods for sensing and correlating signals captured at the bed edge, in the patient and the internation environment for processing medical data and other parameters in real time, such information shared locally and remote.

### BACKGROUND OF THE INVENTION

The current health market consumes at least 9% of the PIB in developed nations, as England and Canada,whose health systems of which are considered more efficient, and up to 17% of the PIB in the Case of the US, where it is estimated that waste generates a detriment on the order of up to 20% of the consumption. In Brazil, health expenses are around 450 billion real, representing 9.2% of the FIB, and the waste is still greater. The expense per capta rotates in R $ 2,2000.00 in the private system and in almost R $ 1.000,00 In the public system. In particular, half of the expense of the operators is directed to the internalization, including the occupation of beds in ICU, which is not well monitored and requires an enormous labor effort of medical personnel. The control of these expenses can therefore be achieved, to have great impact if these shortcomings today can be increasingly solved using automated and intelligent systems that perform the prevention of error and waste and have assisted focus.

Hospitals come over time, computerizing its processes by means of information systems that automate some tasks pertaining to the hospital environment. Most of these systems are directed to management and therefore seeks to reduce the costs and optimization of the administrative processes. Hospitals commonly use some of the following systems: electronic pronunciation; consultation marking; pharmacy control; internalization; laboratory; among others.

The Electronic Medical Records (EMR) or Electronic Hospitals Records (EHR) models are the best known and, while reducing the costs of a hospital unit, around 20-25%, they still fail to perform a more precise monitoring on the patient's health for the proper medical care, since they were born of the administrative and management need. In addition, they are models focused on reports, generating bulky medical records, with repeated and many irrelevant data, are not focused on the specific needs of each patient and do not have good medical usability. More specifically, they are non-automated models of data (the medical data of the monitored patient are read and annotated by the nursing staff at the bedside) and do not reduce the possibility of error, which is the major cost-impact factor operational and process efficiency.

The concepts of automation have long been incorporated into the medical area, and also being used in hospital automation. In this specific context, research has been directed to the development of patient monitoring systems; use of technologies to improve the usability level of the information systems for the medical area; in the definition of network architectures for the transfer of biomedical and data signals, use of devices and sensors; and in the specification of protocols applied to a specific medical area. In a general overview, hospital automation can be observed on two perspectives: information network and control network. The present invention relates to information systems used in the hospital area. The systems used in monitoring patients are used in the second. On the perspective of patient monitoring, the trend is to automate this process by systems using devices and/or sensors to make capture and collection of vital signs of patients. The objective is that the automated collection of signals allows a medical team to be able to carry out medical procedures with increased safety and accuracy.

The present invention therefore proposes a hospital bed automation system that operates in a unique manner, allows monitoring of a patient in the bed by capturing vital signs and other patient-related parameters and the monitoring environment, then performing the collection for proper reading and diagnosis correlation of intelligent mode signals and real-time data processing, database record and transmission for a medical information system, which may be an EMR, EHR or any other system that performs the management of medical information, in order to decrease the error of manual collection inadequate by the health professional and to combine new form medical signals and data. Such information can be shared locally and remote.

### STATE OF THE ART

There are published works on the monitoring of patient's signals from medical equipment and devices in hospital beds and intensive care units (ICU), by more specifically addressing the communication protocol types of the control networks (Dolejs et al, 2004; Murakami et al.. 2006; Varshney, 2006; Godary et al. 2007; valenm et al. 2008) systems based on RFID technology for hospital automation (Florentino et al., 2008) and vital signal monitoring systems for the diagnosis of patients, implementing network communication protocol and software for processing and management of data (Shin et al. 2000). In particular, Vaietm et al (2012) points the state of the art and the architecture of the hierarchy of elements used in hospital automation: systems used in hospital management processes, communication protocols enabling integration between medical devices (hardware) and supervisory systems; fault tolerance logic (system security for ensuring the integrity of patients related to patients). The work is particularly directed to the communication of medical devices (heart rate monitor), pulse oximeter, glucose sensor, serum infusion), located in the hospital bed by means of Ethernet switches or hubs for exchanging data in the network and by means of a supervisory station that monitors the process for maintaining the safety levels of patient data.

PI0603602-3 patent document describes a telemedicine system for remote monitoring of patients in which patient's vital signs are collected from analog or digital medical equipment and devices, with communication between treatment site, monitoring center and remote monitoring by wireless communication. The operation of an interface module establishes automatic communication between server and medical equipment, transforms analog signal into digital and processes the data of the vital signs, generating alarms and reports.

MU8702746-1 patent document describes an electrocardiogram collecting system having an adaptive physical interface device for connection with the ECG or other medical equipment, a man-machine interface for input of specific data of a patient, a portable system embedded ECG collection and a data collection server, with encryption, transmission and reception of the collected and digitized data occurring.

In BR102012004636-9 application, a remote system of specialized medical service also monitors patient vital signs and transmits the data via serial communication with a cellular modem. According to the document, the system collects vital signs of a plurality of medical equipment, and the system can still remotely perform adjustments to medical equipment. However, it does not detail the particular features of system operation.

In WO2015192121, the patient monitoring method has as a differential the creation of a local sensor network that has subsystems and the type of control network communication is cloud based. Transmission of system data occurs through software that generates an electronic order for a patient indicating the parameters to be measured, and the location of the sensor and the patient is determined. The document further reveals that the security protocol operates by HL7 message and the patient identification method occurs by scanning the data to a low energy Bluetooth (BLE) sensor device used as an RFID device. In WO200505511, a method of remote monitoring of data contemplates monitoring, collecting, controlling, transmitting and processing data, but the particular characteristics of each step of the method are not detailed.

US20160026762 details monitoring vital signs and other medical data from various sources using an informational panel module (dashboard) where an on-board software is available, responsible for filtering, analyzing and displaying only relevant information for patient treatment in a customized fashion. In US20160019345 the patient's vital signs are monitored from a telemetry system connected to the patient by sensors, not medical equipment and devices.

Finally, US2015119733 describes a system and method for evaluating an association between a wireless sensor and a monitored patient. The system comprises peripheral electronic devices, each having a communication system and a wireless sensor, each sensor configured to measure a patient's parameter; a central communication point; and processor configured to receive data from the measured parameter and identify the characteristics of the data associated with the patient to establish an association condition between each device and patient. The method establishing such an association condition aims at obtaining a reliable value attributed to the association between each of the sensors and the monitored patient, in order to measure monitoring accuracy.

The prior art therefore presents various systems and methods for automating and monitoring patients local or remotely. The differences between technologies are in the combination of components that constitute such systems, at the interfaces between the acquisition of the signals and the processing and sending of data and methods for processing and evaluation of the monitored data.

In contrast, the present invention proposes a hospital bed automation system in which several signals are captured simultaneously from sensors, medical equipment and devices installed at the bedside, in the patient and in the environment, and collected at interfaces that perform reading and correlation of the signals for processing and logging in database, representing a reading and intelligent interpretation of the data. More particularly, the interfaces that collect said signals are microprocessors containing an embedded computer program that perform intelligent reading methods of each monitored patient. The novelty of the present invention is based on the characteristics of the system that integrates the capture and collection of vital signs, other patient parameters and data from the hospitalization environment (air, lighting, sound, television, blinds, nurse flame, etc.) with data processing, on the interface characteristics (microprocessors) of the hospital bed automation system, as well as the methods performed by the microprocessors to perform reading and correlation of the signals.

The present invention solves the problem of inadequate manual data collection and the excessive amount of data and reports generated on each patient in the hospital environment and thereby reduces the error through more accurate monitoring and performs the reading and correlation of patient signals monitored intelligently, which helps to increase the efficiency of the medical assistance process.

### DETAILED DESCRIPTION OF THE INVENTION

The disclosure of the invention makes reference to the following drawings:
Drawing 1 shows the block diagram of the hospital bed automation system, where the patient's signals and other parameters are captured, collected and sent to the microprocessors (2) and (3) of the bed (1), with reading and correlation occurring of the data processing signals (4), which are transmitted by the connection (14) to a central server (5), which contains a database for recording said data of each patient, an application server (6) that interfaces between the (5) database, a medical information system (7), and local and remote users (8). (2) and (3) have the embedded computer programs (18) and (19), respectively.
Drawing 2 illustrates communication and connection networks (14), (15), (16) and (17) in the bed (1), (14) represent he connections for transmitting the data to the central server (5), outside of (1), (15) and (16) connections between microprocessors (2) and (3), the equipment and medical devices (10) and (11), the edge sensors of the bed and the patient (12) and the ambient sensors (13), and the connections of some of the equipment and medical devices (10) and (11) connected to the patient. (9) Represents a portable device for accessing users. (2) And (3) have the computer programs shipped (18) and (19), respectively. (2) And (3) may or may not be connected by means of (20).
Drawing 3 details methods performed by (18) and (19) embeded in (2) and (3) respectively, since the collection of the signals from the sensors, equipment and devices to the processing of the data, to perform the reading and correlation of signals of (10), (11), (12), and (13), including the perception of uncorrelated signals that generate automatic previous alarms.

The hospital bed automation system, object of the present invention, according to Drawings 1 and 2, comprises: a bed (1) where microprocessors (2) and (3) are installed which collect signals and other medical parameters of a (11), (12) and (13); and wherein the patient is provided with a plurality of sensors, equipment and medical devices installed at the bedside, in the patient and in the inpatient setting. a central server (5) having a database that registers said data of each patient after reading and correlation of the signals for data processing (4); an application server (6) interfacing the database of (5), a system for managing the medical information (7), and local and remote users (8) accessing the information contained in (7) through of (6). According to Drawing 2, the hospital bed automation system further comprises wired and wireless communication networks 14, 15 and 16, 14 being between (2) and (3) and the central server 5 and 15 and 16 between 2 and 3 and the plurality of medical sensors, equipment and devices 10, 11, 12 and 13. Additionally, 17 are connections of the multiparameter apparatus itself that monitor the vital signs of patients from some of the medical devices and devices 10 and 11, such as ECG sensor signals and fans, and 9, are fixed and portable communication devices, such as computers, smartphones and tablets. (9) are used by (8) to access the information in (7) through (6). Finally, (2) and (3) may alternatively be connected by means of (20).

According to Drawings 1 and 2, the operation of the present invention takes place by the acquisition of the signals by (2) and (3), installed in (1), which are captured from (10), (11), (12) and (13). The acquisition of the signals can occur by wired or wireless connection (15) and (16), being integrally collected by (2) and (3) and transmitted by connection with or without wire (14) a (5). Alternatively, the signals at (2) and (3) may be preferably subjected to a reading and correlation step for data processing (4), In order to perform simultaneous reading of signals, correlations between them, identify measurement errors, specific parameters and repeat patterns, to then be transmitted a (5). In (5) the data can be fully stored, permanently or for a period of time Time to be established, which depends on the space available in (5) and the need for storing relevant collection information. The (5) database records said data from each patient, which can be viewed in (9) by means of the application server (6). (6) Interface between the (5) database, the medical information system (7) and local and remote users (8), the patient data Being Transmitted via the Internet and accessed by the user. (7) Comprises any information system or computer program for managing medical information, such as an electronic prontuary, comprising EMR, EHR, or the like, medical intelligence information systems. (8) Accessing medical information by means of a remote or local mode, including devices using touch screen technology. The automation of the bed, proposed by the present invention, allows for the capture and collection of signals, complete automatic reading of bed information, transmission and processing of data in real-time and requires the instrumentation of sensors in the bed and the integration of equipment and medical devices.

Sensors coupled to the bed edge and patient (12) and the ambient sensors (13) comprise: a) sensor for identifying the inclination position of the headboard, coupled to the headboard of the bed; b) patient positioning sensor, which refers to an accelerometer serving as a decubitus sensor, directly coupled to the patient to identify its movement and position; c) diuresis sensor, coupled to the urine collection bag to allow measurement and monitoring of diuresis in real time by flow and by weight; d) patient weight measurement sensor; e) ambient sensors, installed in the patient's hospitalization environment (room or ICU), which allow the measurement of temperature, humidity and luminosity (color and intensity of light), atmospheric pressure and can also control blinds, television, air conditioning and sound, not limited to these. Nursing call, room service and videoconference commands with physicians can still transmit data from the environment through analog-to-digital conversion.

The devices and medical devices (10) and (11) are integrated and connected for real-time visualization, locally and remotely, of the patient's vital signs. This monitoring can occur by digital connection or analog-to-digital conversion, when the equipment or medical device has only analog output. The signals are captured by the connections (17) that are characteristic of the equipment and devices. (10) and (11) include, but are not limited to, multiparameter monitors for ECG, heart rate, respiration, temperature, invasive and non-invasive pressure, pulse oximetry, hemoglobin saturation, ventilator, pump infusion for medication and, alternatively, electrocardiograph, capnograph, among others.

Microprocessors (2) and (3) are small units having embedded intelligence of computer programs that collect the captured signals from (10), (11), (12) and (13) to perform the reading and correlation of these signals, filtering or compressing the data packets and performing processing thereof. (2) And (3) have at least 8 digital and analog inputs and 8 outputs sensors and can contain sensors for capturing the signals from the environment that are custom-enabled, in addition to allowing the coupling of new interfaces, added in modules as needed. Each microprocessor (2) is installed at the inlet of the bed, for example, room or ICU, and processes, via the computer program (18) on board, analog-to-digital conversion and methods that combine the reading of the signals collected from the capture of (10) and (11) to perform correlations between said signals and specific measurements, in addition to identifying measurement errors and repeat patterns, for each patient. Each microprocessor (3) is installed beneath the bed of the patient's bed and processes, via the computer program (19) on board, analog-to-digital conversion, when it is the case, and methods that combine the same. Reading of signals collected from the capture of the fluid, patient and environment sensors (12) and (13) to perform correlations between them and specific measurements, in addition to identifying measurement errors and repeat patterns for each patient. For all measurements, errors and repeat patterns identified by the readings and correlations performed from the signals of (10), (11), (12) and (13) programmed alarms can be generated. Additionally, prior to analog-digital conversion of the captured signals, (18) and (19) they perceive non-correlated signals for generating automatic previous alarms such as, for example, alarm of the pressure monitor when said pressure is too low; alarm risk for dropping when the patient moves in the bed; alarm of the diurease sensor device when the urine collection bag reaches a volume above 2/3

According to Drawing 3, non-limiting examples of methods performed in (2) and (3) by (18) and (19), respectively, to perform (4) are:
Reading the plurality of sensors, equipment and medical devices to perform correlations of of incidence of major patient diagnoses for early detection of disease or infection - for example, low blood pressure, high heart rate, high temperature, and unchanged diuresis in the last 3 hours indicate an infection of the patient;
Reading the plurality of ambient sensors to perform correlations of incidence of secondary diagnosis of the patient—for example, reading the color effect sensor of the ambient light assists in maintaining the lightness in a given range of value, for a given period of time, for patients who have risk of delusions, because this measurement reduces the risk; another example relates to reading the patient's positioning sensor, wherein the change in position of the patient is beneficial, given a given period of time, allowing for the prevention of pressure ulcer; lastly, reading the same head inclination sensor may indicate patient's risk of falling
Signal Reading for measurements developed for a specific purpose, for example, the measurement of diuresis occurs by means of a developed diuresis sensor device, which relates to a digital sensor with sensor coupled to the urine collection bag to allow monitoring flow and volume of urine and the weight of the bag, that is, with volume build-up, the weight of the bag is calculated;
Identification of repetitive patterns (selective signal reading) for example, reading the head positioning sensor only occurs when there is a change in bed slope, rather than reading this sensor in real-time of the same angle without any change;
Identification of measurement errors—for example, reading the patient's positioning sensor due to movement thereof to positions that are not critical; another example would be the Reading of the ECG correlated to the reading of the zero-pulse oximeter, which indicates a disconnection from the sensor and not a patient's health problem;

According to Drawing 2, the connection type (14), (15) and (16) to be established between microprocessors (2) and (3), the central server (5) and the sensors, equipment and medical devices installed in the bed, the patient and the environment (10), (11), (12) and (13) is established be wired or wireless. (14) is established by Ethernet or Wi-fi standard between the microprocessors (2) and (3) and the server (5). In case the medical equipment is analog and has only a serial port, the connection (15) between (2) and (10) and (11) it is via Ethernet or Serial RS232, RS422, RS485 standard. Additionally, the connection (15) between (2) and (10) e (11) may be wireless, comprising Wifi, Xbee, Zigbee, cellular, in case of 10 and 11 transmit digital signal, or, in case the monitoring is remote, in a homecare, for example. The connection (15) can also be established between (3) and (12) by default, Ethernet, serial or wireless.

Connection (16) between (3) and (13) can be by serial, digital or infrared by default. The digital connection is able to inform potential values in mV that are translated into analog values, according to the calibration of each sensor instrumented in the bed, the patient and the environment. (3) can use infrared (16) connection to control the instrumented sensors in the environment, in TV sets, air conditioning using an IR transmitter coupled through a digital connection. Connections (17) do not represent communication networks, but only connections to multi-parametric equipment itself which monitor vital signs of patients from some device and medical devices (10) and (11), such as signals from ECG electric sensors and fans.

According to Drawings 1 and 2, the signals of (10) and (11) captured, read and correlated in (2) data is processed and data is transmitted to (5) by connection (14), by Ethernet or Wi-fi, as well as the signals of (12) and (13) captured, read and correlated by (3) are processed and data is transmitted to (5) by the same connection (14). (2) and (3) perform the transmission, in real-time, from the processed data to (5) through a broadcast communication. Thus, for each bed a different data transmission channel is selected

Data security is established in two ways. In terms of the reliability of the data in the identification of the patient, for (2) being installed at the entrance of the bed (room, ICU) and (3) under the bed of the patient, even if it is transferred, (2) remains in the hospitalization environment and (3) in the bed ensuring the correct identification of the patient's location. In terms of privacy of information, transmission of data is stealthy to not identify the patient. In all cases the system signals the arrival of the data by identifying the microprocessor (2) installed in each hospitalization environment. In the database of (5), which records the medical data and other parameters of each patient, is performed the identification of each patient by each microprocessor (3).

The bed automation system, object of the present invention, can communicate with any other equipment or systems using the HL7 standard, ensuring communication between them and standardizing data exchange that will be accessed by users. It therefore has technical operability, that is, usability and ease of integration with a variety of systems, equipment and medical devices. The present invention has an industrial application and can be employed on medical centers, hospitals and home medical treatment (homecare) allowing complete monitoring of the patient and the more precise control of the process, resulting in more safety of operation of the medical team and patient treatment and reducing operating costs.

1. A hospital bed automation system comprising:
a) a first microprocessor (2) and a second microprocessor (3) installed in a hospital bed (1), wherein the first microprocessor and the second microprocessor are configured to receive a plurality of data comprising vital signs of a patient and other parameters associated with a patient from a plurality of sensors, medical equipment, and devices installed at a rim of the hospital bed.
b) a central server (5) having a database configured to record the plurality of data, the central server configured to read and correlate the plurality of data (4) in real time;
c) an application server (6) configured to interface between the database (5), a system for managing medical information (7), and one or more local and remote users (8) configured to have access to the information contained in the system for managing medical information (7);
d) a plurality of communication networks (14) (15) (16), (17) and (20), wherein:
a first communication network (14) is configured to transmit information from at least one of the first microprocessor (2) or the second microprocessor (3) to the database (5);
a second communication network (15) and (16) configured to collect one or more signals from the plurality of sensors (12) and (13), equipment and medical devices (10), and (11) and convey the one or more signals to at least one of the first microprocessor (2) or the second microprocessor (3);
a network of connections of the equipment and medical devices (10) and (11) configured to monitor one or more of the vital signs of the patient; and
a communication connection (20) between the first microprocessor (2) and the second microprocessor (3); and
e) one or more fixed or portable communication devices (9) used by one or more local or remote users (8) configured to access information from the medical information system (7) via the application server (6)

2. The hospital bed automation system of claim 1, wherein wherein the first microprocessor and the second microprocessor are configured to:
a) acquire one or more of signals from the plurality of sensors (12) and (13) and equipment and medical devices (10) and (11);
b) read and correlate data (4) and transmit the data (4) to the database (5);
c) process and store the data 94) in the database (5);
d) record data from the patient in the database (5) for local or remote access via the Internet, the local or remote user (8) in the medical information system (7) by means of interface with the application server (6) using the fixed or portable device (9).

3. The hospital bed automation system of claim 1, wherein the the first microprocessor (2) and the second microprocessor (3) are small carrier units comprising:
at least 4 digital and analog inputs and outputs; and
one or more added in modules;
wherein the first microprocessor and the second microprocessor are configured to read and correlate the signals received from the plurality of sensors (12) and (13), equipment, and medical devices (10), and (11).

4-5. (canceled)

6. The hospital bed automation system of claim 3, wherein at least one of the first microporcessor or the second microprocessor is configures to generate one or more alarms after reading and correlating the signals.

7. The hospital bed automation system of claim 1, wherein the medical equipment and devices (10) and (11) are connected to one or more local or remote displays, wherein the one or more local or remote displays are configured to display the vital signs of the patient.

8. The hospital bed automation system of claim 7, wherein the medical equipment and device (10) and (11) comprise one or more multimedia monitors for: ECG, heart rate, respiration, temperature, invasive and non-invasive pressure, pulse oximetry, hemoglobin saturation; a ventilator; an infusion pump for a medication; an electrocardiograph; and a capnograph.

9. The hospital bed automation system of 1, further comprising one or more bedside coupled sensors wherein the one or more bedside coupled sensors comprise one or more of a sensor for identifying a position of a bedside of the hospital bed, a patient positioning sensor, a patient weight measurement sensor, and a diuresis sensor.

10. (canceled)

11. The hospital bed automation system of claim 9, wherein the diuresis sensor is configured to monitor a patient's weight and a volume of urine in real time.

12-13. (canceled)

14. The hospital bed automation system of claim 1, wherein the first communication network (14) between at least one of the first microprocessor (2) or the second microprocessor (3) and the central server (5) comprises a standard Ethernet or W-fi or cellular connection.

15. The hospital bed automation system of claim 1, wherein the second communication network (15) is a wired or wireless network.

16. The hospital bed automation system of claim 15, wherein the second communication network is a wire Ethernet or Serial RS-232, RS-422 or RS-485 network, or a wireless W-Fi, Bluetooth, Xbee, or Zigbee network.

17. (canceled)

18. The hospital bed automation system of claim 15, wherein the connection (15) between the second microprocessor (3) and the patient (12) is a wired or wireless connection comprising a digital or analog standard, such as Ethernet, W-fi, Bluetooth, Xbee, Zigbee or Serial RS232, RS422, RS485.

19. The hospital bed automation system of claim 1, characterized by that the second communication network (16) is one or more of a wired or wireless communication network, comprising a serial network, an analog network, a digital network, or an infrared network.

20-22. (canceled)

23. The hospital bed automation system of claim 1, wherein the medical information system (7) is configured to employ one or more systems or programs for medical information management, comprising, electronic medical records (EMR), electronic hospital records (EHR), and medical intelligence information systems.

24. The hospital bed automation system of claim 1, wherein the one or more fixed or portable communication devices (9) comprise one or more fixed computers, portable computers, mobile devices, and tablets which use touch screen.

25. (canceled)

26. A method for performing reading and correlation of signals for real-time data processing, the method comprising:
a) collecting, by one or more microprocessors, data of one or more signals from one or more sensors, devices and medical devices (10), (11), (12) and (13);
b) determining, by the one or more microprocessors, that one or more signals from a single sensor, device or medical device (10), (11), (12) or (13) are uncorrelated;
c) converting, by the one or more microprocessors, the one or more signals from a single sensor, device, or medical devic from an analog format to a digital format;
d) simultaneously reading and correlating, by the one or more microprocessors, the signals of the medical devices (10), (11), (12) and (13);
e) filtering or compacting, by the one or more microprocessors, one or more data packets for transmission across a network; and
f) processing data, by the one or more microprocessors, for transmission to a central server (5).

27. The method for reading and correlating signals for real-time data processing, of claim 26, further comprising generating an automatic pre-alarm, wherein the pre-alarm comprises one or more of: a pressure monitor alarm, a low pressure alarm; an alarm indicative of a risk of a patient falling, when the patient is moving in the bed; and a diuresis sensor alarm when the urine collection bag reaching a volume above two thirds.

28. The method for performing reading and correlation of signals for real-time data processing of claim 26, the method further comprising:
a) simultaneously reading, by one or more microprocessors, the plurality of medical sensors, equipment and medical devices (10), (11), (12) and (13) to perform incidence correlations of major diagnoses of the patient;
b) simultaneously reading, by one or more microprocessors, the plurality of ambient sensors (13) to perform incidence correlations of secondary diagnoses of the patient;
c) measuring, by one or more microporcessors readings from a specialized sensor;
d) identifying, by one or more microprocessors, repetition patterns by selectively reading the signals;
e) identifying, by one or more microprocessors, measurement errors.

29-33. (canceled)

34. The method for performing real-time data reading and correlation for data processing of claim 26 further comprising, transmitting and receiving, by the one or more microprocessors, one or more communication packets using the HL7 standard.

35. (canceled)
